(19) United States
(12) Patent Application Publication
(10) Pub. No.: US 2016/0270940 A1
(43) Pub. Date: Sep. 22, 2016

(54) SOFT ROBOTIC ORTHOPEDIC DEVICE

(71) Applicants: Carl Everett VAUSE, Concord, MA (US); Joshua Aaron Lessing, Cambridge, MA (US); Ryan Richard Knopf, Cambridge, MA (US)

(72) Inventors: Carl Everett VAUSE, Concord, MA (US); Joshua Aaron Lessing, Cambridge, MA (US); Ryan Richard Knopf, Cambridge, MA (US)

(21) Appl. No.: 14/664,695

(22) Filed: Mar. 20, 2015

(51) Int. Cl. A61F 5/01; A61F 5/058; A61B 17/58

(52) U.S. Cl. CPC A61F 5/012; A61B 17/58; A61F 5/013; A61F 5/05816; A61F 5/0585; A61F 5/05858

(57) ABSTRACT

A compact, portable, easily implemented soft robotic orthopedic stabilization device for adjustably and selectively stabilizing a fractured or otherwise injured limb of a patient while providing convenient access to portions of the limb for treatment thereof.

### CROSS-REFERENCE TO RELATED APPLICATIONS

The present application claims the benefit of earlier filed Provisional U.S. Pat. App. No. 61/955,879 by Vause et al. titled “Soft Robotic Orthopedic Device,” filed on Mar. 20, 2014, which is incorporated by reference herein in its entirety and for all purposes.

### FIELD OF THE DISCLOSURE

The disclosure relates generally to the field of orthopedic devices and more particularly to soft robotic orthopedic devices for stabilizing fractured limbs. Numerous methods and devices have been developed for the stabilization and reduction of both open and closed skeletal fractures. One such method, commonly referred to as “external fixation,” involves inserting rigid pins into bone fragments within a patient's limb and then attaching these pins to rigid bars, rings, or other structures that are external to the limb in order to stabilize the fractured bone. Another stabilization method involves surrounding a patient's fractured limb with a pneumatic air cast or splint having one or more inflatable internal air chambers. After such a cast or splint is secured on a limb, the internal air chambers are inflated to expand the device and forcibly stabilize the limb.

These methods and devices are associated with a number of possible shortcomings. For example, external fixation is extremely invasive, is time consuming, and often requires medical imaging devices to accurately target bone fragments for pinning and fixation. Moreover, the devices and equipment that are necessary for performing external fixation take up a great deal of space and are not easily portable. Pneumatic air casts and splints, while relatively small and easy to use, are associated with well-documented complications such as tissue necrosis that may result from over-pressurization of such devices. Pneumatic air casts and splints also restrict access to open wounds. Such access may be necessary or advantageous for administering medical treatments while a limb is stabilized. Moreover, pneumatic air casts and splints do not provide adjustable, targeted pressure to particular areas on a patient's limb.

In view of the foregoing, it would be advantageous to provide an orthopedic stabilization device that is compact, lightweight, and easily portable when not in use. It would further be advantageous to provide such a stabilization device that provides convenient access to a limb that is stabilized therewith. It would further be advantageous to provide such a stabilization device that facilitates the application of adjustable pressure to targeted areas of a stabilized limb.

### BRIEF DESCRIPTION OF THE DRAWINGS

FIG. 1 is perspective view illustrating an exemplary embodiment of a soft robotic orthopedic device in accordance with the present disclosure.

FIG. 2 is a cross sectional view illustrating an exemplary embodiment of a rigidizable material in accordance with the present disclosure.

FIG. 3a is a perspective view illustrating an exemplary embodiment of another soft robotic orthopedic device in accordance with the present disclosure.

FIG. 3b is a perspective view illustrating an exemplary embodiment of a spine of the soft robotic orthopedic device shown in FIG. 3a.

FIG. 3c is a front view illustrating an exemplary embodiment of an actuator of the soft robotic orthopedic device shown in FIG. 3a.

FIG. 3d is a side view illustrating the actuator shown in FIG. 3c.

FIG. 3e is a side view illustrating an alternative embodiment of an actuator of the soft robotic orthopedic device shown in FIG. 3a.

FIG. 3f is a side view illustrating another alternative embodiment of an actuator of the soft robotic orthopedic device shown in FIG. 3a.

FIG. 4a is a perspective view illustrating an exemplary embodiment of another soft robotic orthopedic device in accordance with the present disclosure in an undeployed state.

FIG. 4b is a side view illustrating an exemplary embodiment of a foam mixer/ejector of the soft robotic orthopedic device shown in FIG. 4a.

FIG. 4c is a perspective view illustrating the soft robotic orthopedic device shown in FIG. 4a in a deployed state.

### SUMMARY

“Soft robotic” actuators that are configured to perform new fundamental motions—such as bending, twisting, and straightening—are described. Soft robotic technologies are discussed in PCT International Publication Number WO2012/148472, which is incorporated herein by reference in its entirety. The present invention includes the implementation of soft robotic technologies into specific configurations that are useful as orthopedic devices, and related methods that employ such soft robotic configurations.

Certain embodiments of the present disclosure describe fabrication and operation of pressurizable networks of channels or chambers (Pneu-Nets) embedded in elastomeric or extensible bodies. The pressurizable network actuators can be programmed to change shape and mechanical properties using an external stimulus, including pneumatic or hydraulic pressure. The soft robot structures utilize designs of embedded pneumatic or hydraulic networks of channels in elastomers that inflate like balloons for actuation or in folded extensible fabrics that can open up when pressurized. A pluralities of chambers embedded within an elastomer can be used as a series of repeating components. Stacking and connecting these repeated components provide structures capable of complex motion. In this type of design, complex motion requires only a single pressure source (although more than one source can be used, if desired). The appropriate distribution, configuration, and size of the pressurizable networks, in combination with a sequence of actuation of specific network elements, determine the resulting movement.

In one aspect, a soft body robotic device includes a flexible molded body having a plurality of interconnected chambers disposed within the molded body. A portion of the molded body is comprised of an elastically extensible material and a portion of the molded body is strain limiting relative to the elastically extensible material. The soft body robotic device further includes a pressurizing inlet that is configured to receive fluid for the plurality of interconnected chambers. The molded body in the soft body robotic device is configured to preferentially expand when the plurality of interconnected chambers are pressurized by the fluid, causing a bending motion around the strain limiting portion of the molded body.

In another aspect, a soft body robotic device includes a flexible molded body comprising a plurality of interconnected pleated chambers. The flexible molded body includes a flexible material and is affixed to a strain limiting member in such a manner that the strain limiting member forms a wall of the plurality of interconnected pleated chambers. The thickness of the molded body is at least 1 mm. The soft body robotic device further includes a pressurizing inlet that is configured to receive fluid for the plurality of interconnected pleated chambers. The plurality of interconnected pleated chambers are configured to preferentially unfold when the flexible molded body is pressurized through the pressurizing inlet, causing bending motion around the strain limiting member.

In yet another aspect, a soft body robotic device is capable of extension. This soft robotic device includes a flexible molded body having a plurality of interconnected chambers disposed within the molded body. The soft robotic device also includes a sealing member in a facing relationship with the flexible molded body, in which the flexible molded body and the sealing member together define a plurality of channels. Each channel is defined by upper, lower and side walls. The sealing member is in a state of compression in its resting state. The soft robotic device additionally includes a pressurizing inlet in fluid communication with the plurality of channels. The plurality of channels are positioned and arranged such that the soft body robotic device expands to relieve the strain in the sealing member when the soft body robotic device is pressurized through the inlet.

In another aspect, the present invention relates to orthopedic devices that include actuators having a flexible body with multiple interconnected chambers and comprising a first elastic portion and a second strain limiting portion, along with a spine having a connection point for removably attaching the actuator and a fluid inlet in communication with the interconnected chambers of the flexible body, which inlet can connect to a fluid source for controlling a pressure within the chambers, where increasing that pressure causes the first portion of the flexible body to bend about the second portion of the flexible body, thereby changing the shape of the actuator. Various embodiments may utilize flexible bodies with multiple elastic (or “first”) and/or strain limiting (“second”) portions arranged so that increasing the pressure in the chambers causes the actuator to assume a shape having multiple bends and/or a compound bend. The actuators are, in some instances, transversally attached to the spine in a longitudinally spaced relationship. The spine, which optionally includes a collapsible body with an interior fluid chamber such that a rigidity of the spine varies with a pressure in the interior chamber, can also include a quick disconnect valve for coupling to the quick disconnect valve; when connected, the valve is placed in fluid communication with the interconnected chambers of the actuator. The spine can also include a fluid source for connecting to the fluid, which fluid source controllably alters the pressure in the chambers and causes the actuator to change shape. In some cases, the device includes a fluid source connectable to the fluid inlet, and optionally includes a plurality of actuators, in which case the fluid source optionally connects to each actuator independently.

In yet another aspect, the present invention relates to an orthopedic device that includes a spine and an actuator formed at least partly of an elastomeric material which has a pressurizable internal fluid compartment, which actuator is affixed to the spine and configured to stabilize a limb of a patient when pressurized and to form a flexible, collapsible configuration when depressurized. The device optionally includes a supplemental beam for applying additional stabilization to the limb, which beam is affixed to the actuator and optionally has a collapsible body with an interior fluid chamber, where the rigidity of the supplemental beam varies with the pressure in the chamber. The spine is, optionally or additionally, physically deformable to conform to a counter of the patient's limb. The device also optionally includes multiple actuators, which are independently pressurizable, and may also include a foam mixer/ejector fluidly communicating with the internal fluid compartment, while the actuator may also include a membrane configured to expand away from a limb of the patient, thereby limiting the amount of pressure applied to the patient.

In yet another aspect, the invention relates to a method of treating a patient that includes contacting the limb of a patient with an orthopedic device such as those described above. The method can also include coupling a fluid source to the fluid inlet, and altering the pressure, selectively, within the chambers, and/or it can include conforming the spine to a contour of the limb, and/or positioning the spine adjacent to the limb extending along at least a portion of the limb. The actuators are optionally placed transversely relative to the limb. The method can also include a step of pressurizing the plurality of interconnected chambers, causing the actuator to at least partially wrap around the limb thereby stabilizing it.

### DETAILED DESCRIPTION OF THE PREFERRED EMBODIMENTS

The present invention will now be described more fully hereinafter with reference to the accompanying drawings, in which preferred embodiments of the invention are shown. The invention, however, may be embodied in many different forms and should not be construed as being limited to the embodiments set forth herein. Rather, these embodiments are provided so that this disclosure will be thorough and complete, and will fully convey the scope of the invention to those skilled in the art. In the drawings, like numbers refer to like elements throughout.

In accordance with the present disclosure, a compact, portable, “soft robotic” orthopedic stabilization device is provided for adjustably stabilizing a fractured or otherwise injured limb of a patient while providing convenient access to portions of the limb.

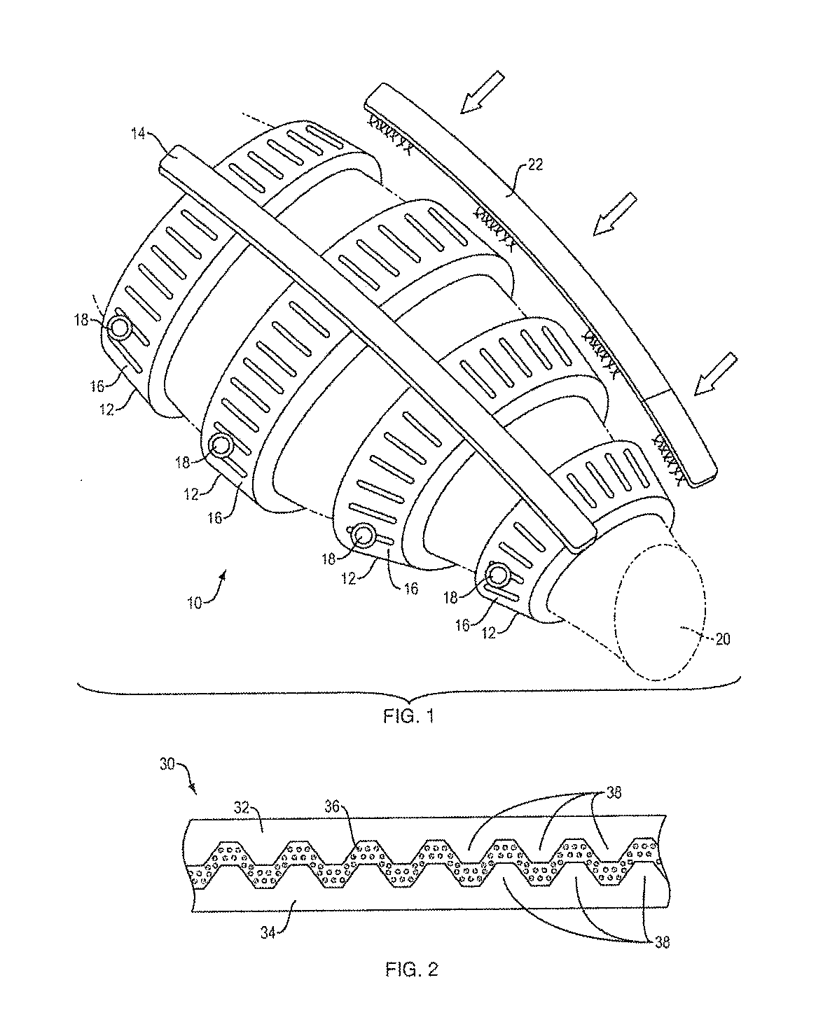

Referring to FIG. 1, an exemplary stabilization device 10 (hereinafter “the device 10”) in accordance with the present disclosure is shown. The device 10 may include a plurality of elongated actuators 12 that are transversally attached to a rigid spine 14 in a longitudinally-spaced relationship. The spine 14 may be formed of any suitably rigid, durable material, including, but not limited to, metal, plastic, wood, and various composites. The spine 14 may alternatively be formed of a flexible, collapsible body having an interior fluid chamber that may be pneumatically or hydraulically pressurized for rigidizing the spine 14 during use. In some embodiments, the spine 14 may be plastically deformable, such as may be advantageous for conforming the spine 14 to the general contour of a limb that is stabilized by the device 10.

Each of the actuators 12 may be defined by a flexible body 16 having one or more pressurizeable (e.g., inflatable) fluid channels and/or chambers formed therein. A portion of the flexible body 16 may be formed of an elastomeric material and another portion of the flexible body 16 may be strain limiting relative to the elastomeric material. The elastomeric portions of the flexible body 16 may be caused to bend around the strain limiting portions via pressurization and depressurization of the fluid chambers, thereby allowing the actuators 12 to be controllably expanded, contracted, shaped, and rigidized in a predefined manner as further described below. The device 10 is shown as having four actuators 12, but it is contemplated that more or fewer actuators 12 may be implemented without departing from the present disclosure. It is further contemplated that the actuators 12 may be removably attached to the spine 14 and that the actuators 12 may therefore be attached to, and removed from, the spine 14 as desired.

The fluid chambers in each of the actuators 12 may be in fluid communication with a pressurizing fluid inlet 18 located on an exterior of each actuator 12. One or more pressurizeable fluid sources (not shown) may be simultaneously or sequentially connected to each of the pressurizing fluid inlets 18 for independently and controllably pressurizing and/or depressurizing each of the actuators 12. Suitable pressurizeable fluid sources may include any type of pneumatic or hydraulic fluid pressurization device, including, but not limited to, manually-operated pumps/bulbs, electric air compressors, compressed gas cartridges, etc. The pressurizeable fluid source may further include, or may be coupled to, a safety blow off valve or an electromechanical device with a pressurized fluid reserve which may be controllably released to maintain a specific pressure in the actuators 12, such as for preventing over-pressurization thereof. Alternatively, the actuators 12 may be provided with a membrane that is configured to expand away from a limb to prevent the application of excessive pressure.

In order to use the device 10, the spine 14 may be positioned adjacent to a fractured or otherwise injured limb 20 of a patient, such as with the spine 14 extending longitudinally along the limb 20, and with the depressurized actuators 12 positioned in transverse engagement with, and/or loosely draped on or loosely wrapped around, a portion of the limb 20 that requires stabilization. The pressurizeable fluid source (described above) may then be actuated to simultaneously or sequentially pressurize the actuators 12, causing the actuators 12 to expand, wrap around, and rigidly grip the limb 20 as shown in FIG. 1. As described above, the configuration of the actuators 12, including the positions, configurations, and orientations of the internal fluid chambers and elastomeric and strain limiting materials of the actuators 12, causes the actuators 12 to move in such a predefined manner and to take on such a predefined configuration when pressurized. The rigidized device 10 thereby securely stabilizes the limb 20. Moreover, since each of the actuators 12 may be pressurized independently, force may be controllably and selectively applied to specific areas of the secured limb 20 by varying the degrees to which each of the actuators 12 is pressurized. The device 10 may thereby be snugly conformed to the various contours of a secured limb without applying excessive force to portions thereof.

Still referring to FIG. 1, one or more supplemental beams 22 may be affixed to the rigidized actuators 12 for providing additional support and stabilization to the limb 20. Like the spine 14, the supplemental beam 22 may be formed of any suitably rigid, durable material, including, but not limited to, metal, plastic, wood, and various composites. Alternatively, the supplemental beam 22 may be formed of a flexible, collapsible body having an interior fluid chamber that may be pneumatically or hydraulically pressurized for rigidizing the supplemental beam 22 during use. In some embodiments, the supplemental beam 22 may be plastically deformable, such as may be advantageous for conforming the supplemental beam 22 to the general contour of a limb that is stabilized by the device 10. The supplemental beam 22 may be connected to one or more of the actuators 12 using any suitable connective means, including, but not limited to, adhesives, mechanical fasteners, hook-and-loop fasteners, etc.

As an alternative to the pressurizeable embodiments of the actuators 12, the spine 14, and the supplemental beam 22 described above, it is contemplated that some or all of these components may instead be formed of a “vacuum activated” or “pressure activated” rigidizing material 30, a cross sectional view of which is illustrated in FIG. 2. The material 30 may be formed of two layers 32, 34 of an elastomeric material (or a composition of rigid and elastomeric material) separated by a layer 36 of open cell foam. The layers 32, 34 may have inwardly facing surfaces that are provided with complementary surface features, such as the teeth 38 shown in FIG. 2. Many other complementary surface features are contemplated and may be similarly implemented without departing from the present disclosure. The layer 36 may be in fluid communication with a vacuum port (not shown), such as may be located on an exterior of a component that is formed of the material 30. In the expanded configuration of the material shown in FIG. 2, the material layers 32, 34 are able to flex and shift alongside each other, and the material is generally pliable and foldable. However, when a vacuum is applied to the layer 36, such may be achieved by connecting a vacuum source (e.g., an air pump) to the above-described vacuum port, the layer 36 may be collapsed and the complementary teeth 38 of the layers 32, 34 may firmly interdigitate. The material 30 thereby rigidizes and is no longer pliable or foldable. The actuators 12, spine 14, and/or supplemental beam 22 may therefore be implemented using the material 30 to provide on-the-spot rigidization of a support device for stabilizing a fractured limb in a manner similar to that described above. An alternative embodiment of the material 30 is contemplated in which a layer of fluid is provided in a space between the layers 32, 34, wherein such fluid may be shunted out of the space upon adverse deflection of a patient's limb, thereby causing the layers 32, 34 to lock together and rigidize as described above.

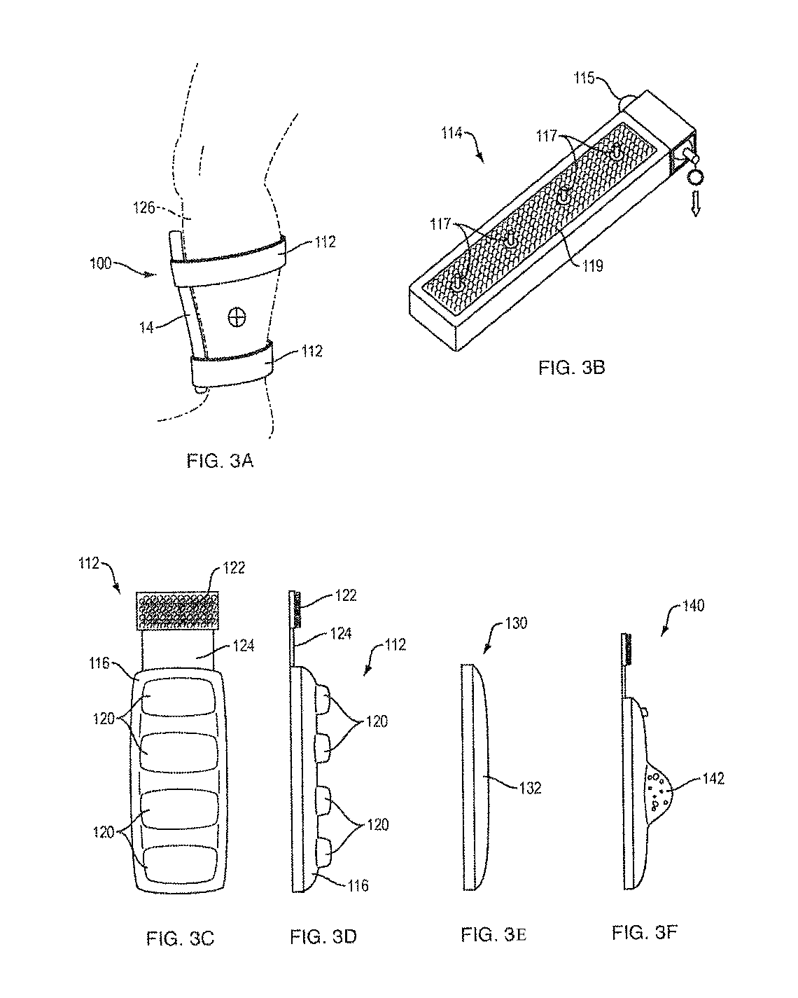

Referring to FIG. 3a, a stabilization device 100 is shown that represents a non-limiting, exemplary variant of the device 10 described above. The device 100 may include actuators 112 that are generally similar to the actuators 12 described above and a spine 114 that is generally similar to the spine 14 described above. Referring to FIG. 3b, the spine 114 may include a controllably-actuated, pressurized fluid source 115 (e.g., a pull-pin CO₂ cartridge) that is in fluid communication with one or more quick disconnect valves 117 (e.g., magnetic quick disconnect valves) located on an exterior of the spine 114. An exterior surface of the spine 114 may further be provided with Velcro 119 or other connective means for facilitating attachment of the actuators 112 as further described below. Referring to FIGS. 3c and 3d, each of the actuators 112 may be defined by a flexible body 116 having a plurality of pressurizeable (e.g., inflatable) fluid channels and/or chambers 120 formed therein. Each actuator 112 may further be provided with a piece of Velcro 122 or other connective means attached to an end of the flexible body 116 by an elastic band 124.

In order to use the device 100, the spine 114 may be positioned adjacent to a fractured or otherwise injured limb 126 of a patient, such as with the spine 114 extending longitudinally along the limb 126 as shown in FIG. 3a. Each of the depressurized actuators 112 may then be attached to one of the quick disconnect valves 117 of the spine 114, thereby placing the fluid chambers 120 of the actuators 112 in fluid communication with the pressurized fluid source 115. The actuators 112 may then be wrapped around a portion of the limb 126 that requires stabilization, with the actuators 112 secured to the spine 114 using the Velcro 119 and 122. The pressurized fluid source 115 may then be actuated to pressurize the actuators 112, causing the actuators 12 to expand and rigidly grip the limb 126. The rigidized device 100 thereby securely stabilizes the limb 126.

Referring to FIG. 3e, an exemplary alternative actuator 130 of the device 100 is shown that includes a single, contiguous pressurizeable fluid chamber 132. Such a configuration may be advantageous for constricting blood flown in a limb when the actuator 130 is rigidized.

Referring to FIG. 3f, an exemplary alternative actuator 140 of the device 100 is shown having an interior surface that is impregnated with a blood clotting agent 142 (e.g., Quick Clot) that may be brought into contact with a patient's wound when the actuator 140 is rigidized.

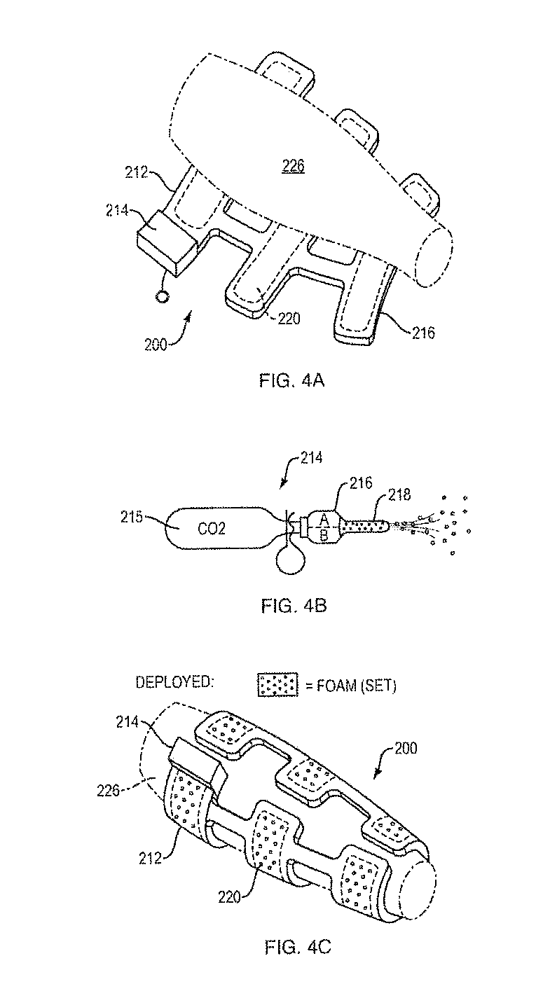

Referring to FIG. 4a, a stabilization device 200 is shown that represents another non-limiting, exemplary variant of the device 10 described above. The device 200 may include a “monolithic” (i.e., one-piece) actuator 212 defined by a flexible body 216 having a size and shape that are adapted to be wrapped around a limb 226 of a patient. The flexible body 216 may define one or more interconnected fluid channels and/or chambers 220 formed therein. The actuator 212 may be provided with an integrated foam mixer/ejector 214 that is in fluid communication with the fluid chambers 220. Referring to FIG. 4b, the foam mixer/ejector 214 may include a controllably-actuated, pressurized fluid source 215 (e.g., a pull-pin CO₂ cartridge) that is coupled to a reservoir 216 containing an unmixed, two-part foam (e.g., a polyurethane foam), the reservoir 216 being connected to a turbulent mixing nozzle 218. The two-part foam may be adapted to harden or “set” after the foam is mixed and expelled by the nozzle 218 as further described below.

In order to use the device 200, the actuator may be wrapped around a fractured or otherwise injured limb 226 of a patient as shown in FIG. 4c. The pressurized fluid source 215 of the foam mixer/ejector 214 may then be actuated, causing the two-part foam in the reservoir 216 to be mixed and expelled by the turbulent mixing nozzle 218. The mixed foam may thereby fill the fluid chambers 220 of the actuator 212. After a period of time (e.g., 30-60 seconds), the mixed foam in the actuator 212 may set, thereby rigidizing the actuator 212 and securely stabilizing the limb 226.

In view of the forgoing, it will be appreciated that the disclosed devices 10, 100, and 200 employ externally attached soft robotic structures to achieve stabilization of an injured limb without requiring the insertion of pins into fractured bone fragments. The devices 10, 100, and 200 are therefore less invasive, more tissue-friendly, and can be implemented more quickly and easily than traditional external fixation devices. Moreover, since embodiments of the devices 10, 100, and 200 include actuators that may be independently pressurized, the amount of force applied to specific areas of a limb can be controlled to mitigate the risk of over-pressurization and resulting tissue damage relative to traditional pneumatic casts and splints. Still further, the devices 10, 100, and 200 have generally open structures that provide many points of access (e.g., in between actuators) to open wounds for administering medical treatment. Still further, when the devices 10, 100, and 200 are not in use (i.e., when the devices are depressurized), their actuators are flexible and collapsible for convenient storage and transport.

It is contemplated that the devices 10, 100, and 200 may be used intra-operatively to reduce a fracture and to align bone fragments during a definitive fixation procedure such as plating, rodding, or nailing. Since the devices 10, 100, and 200 may be formed entirely of non-metallic materials, they may be MRI compatible and X-ray translucent.

It is contemplated that embodiments of the devices 10, 100, and 200 may be configured to facilitate manual or automatic alternating, pulsating, or modulating of the amount of pressure that is applied to an area or set of areas of a limb that is secured therewith over time such that no single section of soft tissue experiences a detrimental level of pressure application while simultaneously maintaining functionality of the device.

Embodiments of the devices 10, 100, and 200 are contemplated in which the devices 10, 100, and 200 may be integrated into a sock, sleeve, cuff, or other similar garment or structure to be used as a simple splint for less severe injuries, such as sports sprains, or for providing support during post-surgical rehabilitation. Still further, by combining the actuators of the various embodiments with pneumatic or hydraulic control systems, the devices 10, 100, and 200 may be implemented as a post-operative assist or physical therapy rehabilitation devices.

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

While the present invention has been disclosed with reference to certain embodiments, numerous modifications, alterations and changes to the described embodiments are possible without departing from the sphere and scope of the present invention, as defined in the appended claim(s). Accordingly, it is intended that the present invention not be limited to the described embodiments, but that it has the full scope defined by the language of the following claims, and equivalents thereof.

What is claimed is:

1. A orthopedic device comprising:
an actuator comprising a flexible body with a plurality of interconnected chambers disposed therein, the flexible body comprising a first relatively elastic portion, and a second relatively strain limiting portion;
a spine comprising a connection point for removably attaching the actuator; and
a fluid inlet in fluid communication with the plurality of interconnected chambers of the flexible body, the fluid inlet configured to couple to a fluid source for controlling a pressure within the plurality of interconnected chambers, wherein increasing the pressure causes the first portion of the flexible body to bend around the second portion of the flexible body, thereby causing the actuator to change shape.

2. The orthopedic device of claim 1, the flexible body comprising a plurality of first portions and a plurality of second portions, wherein increasing the pressure causes the actuator to assume a shape including at least one of a plurality of bends or a compound bend.

3. The orthopedic device of claim 1, comprising a plurality of actuators transversally attached to the spine in a longitudinally-spaced relationship.

4. The orthopedic device of claim 1, the spine comprising a collapsible body with an interior fluid chamber, wherein a rigidity of the spine varies with a pressure within the interior fluid chamber.

5. The orthopedic device of claim 1, the spine comprising
a quick disconnect valve for coupling with the fluid inlet, wherein, upon coupling, the quick disconnect valve is in fluid communication with the plurality of interconnected chambers; and
a fluid source in fluid communication with the quick disconnect valve, wherein the fluid source controllably alters the pressure in the plurality of chambers, thereby causing the actuator to change shape.

6. The orthopedic device of claim 1, further comprising a fluid source for connecting to the fluid inlet to alter the pressure in the plurality of interconnected chambers of the actuator.

7. The orthopedic device of claim 6, comprising a plurality of actuators, the fluid source configured to independently couple with each of the plurality of actuators for controllably altering the pressure in the plurality of interconnected chambers of a coupled actuator.

8. An orthopedic device, comprising:
a spine; and
an actuator formed at least partly of an elastomeric material and having a pressurizeable internal fluid compartment, wherein the actuator is affixed to the spine and configured to wrap around and stabilize a limb of a patient when the internal fluid compartment is pressurized and to assume a flexible, collapsible configuration when the internal fluid compartment is depressurized.

9. The orthopedic device of claim 8, comprising a supplemental beam for applying additional stabilization to the limb, wherein the supplemental beam is affixed to the actuator.

10. The orthopedic device of claim 9, the supplemental beam comprising a collapsible body with an interior fluid chamber, wherein a rigidity of the supplemental beam varies with a pressure within the interior fluid chamber.

11. The orthopedic device of claim 8, wherein the spine is plastically deformable for conforming to a contour of a limb.

12. The orthopedic device of claim 8, comprising a plurality of actuators, wherein the plurality of actuators are independently pressurizeable for selectively applying force to one or more specific areas of the limb.

13. The orthopedic device of claim 8, the actuator comprising a foam mixer/ejector, wherein the foam mixer/ejector is in fluid communication with the pressurizeable internal fluid compartment.

14. The orthopedic device of claim 8, the actuator comprising a membrane, the membrane configured to expand away from the limb of the patient, the membrane for limiting an amount of pressure applied to the limb of the patient.

15. A method for treating a patient, comprising the steps of:
contacting a limb of the patient with an orthopedic device, comprising:
an actuator comprising a flexible body with a plurality of interconnected chambers disposed therein, the flexible body comprising a first relatively elastic portion, and a second relatively strain limiting portion;
a spine comprising a connection point for removably attaching the actuator; and
a fluid inlet in fluid communication with the plurality of interconnected chambers of the flexible body, the fluid inlet configured to couple to a fluid source for controlling a pressure within the plurality of interconnected chambers, wherein increasing the pressure causes the first portion of the flexible body to bend around the second portion of the flexible body, thereby causing the actuator to change shape.

16. The method of claim 15, further comprising the steps of:
coupling a fluid source to the fluid inlet; and
altering, selectively, the pressure within the plurality of interconnected chambers.

17. The method of claim 15, further comprising the step of conforming the spine to a contour of the limb.

18. The method of claim 15, further comprising the step of positioning the spine adjacent to the limb, with the spine extending longitudinally along at least a portion of the limb.

19. The method of claim 15, further comprising the step of positioning the actuator in transverse engagement with the limb.

20. The method of claim 15, further comprising the step of pressurizing the plurality of interconnected chambers of the actuator, the pressurizing causing the actuator to at least partially wrap around the limb, thereby stabilizing it.
